(19) United States
(12) Patent Application Publication
(10) Pub. No.: US 2020/0291490 A1
(43) Pub. Date: Sep. 17, 2020

(54) Risk Stratification for Contagious Disease

(71) Applicant: SENSIVA HEALTH LLC, New Orleans, LA (US)

(72) Inventors: Tarun Jolly, New Orleans, LA (US); James Silliman, Georgetown, SC (US); David Vigerust, Nashville, TN (US)

(73) Assignee: SENSIVA HEALTH LLC, New Orleans, LA (US)

(21) Appl. No.: 16/855,046

(22) Filed: Apr. 22, 2020

Related U.S. Application Data
(60) Provisional application No. 63/005,409, filed on Apr. 5, 2020.

(51) Int. Cl. C12Q 1/70; G01N 33/68; G16H 10/40; G16H 50/30; G16H 10/60

(52) U.S. Cl. CPC C12Q 1/70; G01N 33/6854; G16H 10/40; G16H 50/80; G16H 10/60; G01N 2800/26; C12Q 2600/112; G16H 50/30

(57) ABSTRACT

A method of risk stratification for contagious disease is provided. The method performs a reverse transcriptase polymerase chain reaction (RT-PCR) test on a patient to determine the presence of a viral infection. The method next performs tests for the presence of antibody Immunoglobulin M (IgM) and Immunoglobulin G (IgG) assays. The method assigns the patient a level of readiness to return to society corresponding to the combination of the results of the qualitative RT-PCR, IgM, and IgG tests and quantitative IgG+ antibody testing. The levels of readiness to return to society may be verified by a QR code on a mobile device.

### CROSS-REFERENCE TO RELATED APPLICATIONS

This application claims priority to U.S. Provisional Patent Application No. 63/003,795, filed Apr. 1, 2020, and U.S. Provisional Patent Application No. 63/005,409, filed Apr. 5, 2020, the entirety of each application is incorporated by reference as if fully disclosed herein.

### BACKGROUND OF THE INVENTION

### I. Field of the Invention

The present invention relates generally to a method of risk stratification for contagious disease.

### II. General Background

During the COVID-19 Pandemic, the healthcare diagnostic testing industry has struggled to come up with a uniform response. As healthcare systems and providers are finding themselves increasingly taxed among a growing fear among citizens, the laboratory diagnostic response has been to create testing and make it available to simply diagnose COVID-19. Although this is a critical and important first step, a more coordinated response is required. As such, the present invention discloses a proprietary algorithm of testing patients using a combination of available diagnostics, public health data, and eventual vaccination data to risk stratify individuals and have them able to return to the workforce and begin to interact normally with others while still minimizing the future impact of possible reinfection.

Studies and publications have been published for pandemics such as SARS-Cov1 and MERS. These articles are incorporated by reference as if disclosed fully herein:
Gorse G J, Patel G B, Vitale J N, O'Connor T Z. Prevalence of antibodies to four human coronaviruses is lower in nasal secretions than in serum. Clin Vaccine Immunol 2010; 17:1875-1880.
Yeh K M, Chiueh T S, Siu L K, Lin J C, Chan P K, Peng M Y, Wan H L, Chen J H, Hu B S, Perng C L, Lu J J, Chang F Y. Experience of using convalescent plasma for severe acute respiratory syndrome among healthcare workers in a Taiwan hospital. J Antimicrob Chemother 2005; 56:919-922.
Huang L R, Chiu C M, Yeh S H, Huang W H, Hsueh P R, Yang W Z, Yang J Y, Su I J, Chang S C, Chen P J. Evaluation of antibody responses against SARS coronaviral nucleocapsid or spike proteins by immunoblotting or ELISA. J Med Virol 2004; 73:338-346.
Lee N, Chan P K, Ip M, Wong E, Ho J, Ho C, Cockram C S, Hui D S. Anti-SARS-CoV IgG response in relation to disease severity of severe acute respiratory syndrome. J Clin Virol 2006; 35:179-184.
Shen C, Wang Z, Zhao F, Yang Y, Li J, Yuan J, Wang F, Li D, Yang M, Xing L, Wei J, Xiao H, Yang Y, Qu J, Qing L, Chen L, Xu Z, Peng L, Li Y, Zheng H, Chen F, Huang K, Jiang Y, Liu D, Zhang Z, Liu Y, Liu L. Treatment of 5 Critically Ill Patients With COVID-19 With Convalescent Plasma. JAMA 2020.
Alshukairi A N, Khalid I, Ahmed W A, Dada A M, Bayumi D T, Malic L S, Althawadi S, Ignacio K, Alsalmi H S, Al-Abdely H M, Wali G Y, Qushmaq I A, Alraddadi B M, Perlman S. Antibody Response and Disease Severity in Healthcare Worker MERS Survivors. Emerg Infect Dis 2016; 22.
Liu W, Fontanet A, Zhang P H, Zhan L, Xin Z T, Basil L, Tang F, Lv H, Cao W C. Two-year prospective study of the humoral immune response of patients with severe acute respiratory syndrome. J Infect Dis 2006; 193:792-795.
Tang F, Quan Y, Xin Z T, Wrammert J, Ma M J, Lv H, Wang T B, Yang H, Richardus J H, Liu W, Cao W C. Lack of peripheral memory B cell responses in recovered patients with severe acute respiratory syndrome: a six-year follow-up study. J Immunol 2011; 186:7264-7268.

### SUMMARY OF INVENTION

In accordance with embodiments of the invention, a method of risk stratification for contagious disease is provided. The method includes a step (a) of performing a reverse transcriptase polymerase chain reaction (RT-PCR) test on a patient to determine the presence of a viral infection. The method includes a step (b) of performing qualitative tests for the presence of antibody Immunoglobulin M (IgM) and Immunoglobulin G (IgG) assays. In step (c), if the RT-PCR test in step (a) is negative and the IgM and IgG tests in step (b) are negative, the method informs the patient that the patient has not been exposed to the virus, has not built any antibodies, does not have immunity, should not return to society, and repeat step (a) and step (b) every two weeks. In step (d), if the RT-PCR test in step (a) is positive and the IgM and IgG tests in step (b) are negative, the method informs the patient that the patient is contagious, does not have immunity, has not produced antibodies, and moves to step (i). In step (e), if the RT-PCR test in step (a) is positive, the IgM test in step (b) is positive, and the IgG test in step (b) is negative, the method informs the patient that the patient is contagious, has begun producing antibodies, does not have immunity, and moves to step (i). In step (f), if the RT-PCR test in step (a) is positive and the IgM and IgG tests in step (b) are positive, the method informs the patient that the patient is potentially contagious, has begun producing antibodies, and performs steps (j) through (l). In step (g), if the RT-PCR test in step (a) is negative, the IgM test in step (b) is positive, and the IgG test in step (b) is negative, the method informs the patient that the patient has been exposed to the virus, is not contagious, has begun producing antibodies, does not have immunity, and performs step (i). In step (h), if the RT-PCR test in step (a) is negative and the IgM and IgG tests in step (b) are positive, the method informs the patient that the patient has been exposed to the virus, is not contagious, and performs steps (j) through (l). The method includes a step (i) of recommending isolation for the patient for a first period of time, and repeating steps (a) through (b) upon the conclusion of the first period of time. The method includes a step (j) of performing quantitative testing the patient's level of titers of IgG. In step (k), if the patient's level of IgG titers tested in step (j) is low, the method recommends the patient refrain from reentry into society for a second period of time to allow for further production of IgG antibodies, and repeat steps (j) through (l) upon the conclusion of the second period of time. In step (l), if the patient's level of IgG titers tested in step (j) is high, the method informs the patient that the patient is immune, and recommends the patient return to society.

In one embodiment, the method further includes the step of testing the patient's IgM and IgG levels at three-month intervals from the return to society to track immunity.

In another embodiment, the viral infection is the novel coronavirus that causes COVID-19.

In yet another embodiment, the patient is assigned in steps (c) through (h) a color and status level number indicate level of readiness to return to society.

In one embodiment, the color and status level number are displayed on a mobile device through a mobile application.

In another embodiment, the color and status level number are verified through a QR code displayed through the mobile application.

In yet another embodiment, the patient is assigned the color yellow and status level number 1 in step (c).

In one embodiment, the patient is assigned the color red and status level number 2 in step (d).

In another embodiment, the patient is assigned the color orange and status level number 3 in step (e).

The method of claim 4, wherein the patient is assigned the color orange and status level number 3 in step (f) and remains level 3 until the PCR negative.

In one embodiment, the patient is assigned the color blue and status level number 4 in step (g).

In another embodiment, the patient is assigned the color green and status level number 5 in step (d).

In another embodiment, the patient is assigned the colors red, white, and blue and status level number 6 in step (l).

In yet another embodiment, the low level of titers in step (k) is below 640 mg/dL.

In one embodiment, the high level of titers in step (l) is above 640 mg/dL.

### BRIEF DESCRIPTION OF THE DRAWINGS

The foregoing and other objects, features, and advantages of the invention are apparent from the following detailed description taken in conjunction with the accompanying drawings in which like parts are given like reference numerals and, wherein:

FIG. 1 depicts a flow chart of a method of risk stratification for contagious disease in accordance with embodiments of the present invention.

FIG. 2 depicts a flow chart a PCR positive algorithm of a method of risk stratification for contagious disease in accordance with embodiments of the present invention.

FIG. 3 depicts a flow chart a PCR negative algorithm of a method of risk stratification for contagious disease in accordance with embodiments of the present invention.

FIG. 4 depicts a level and smart phone verification mechanism in accordance with embodiments of the present invention.

The images in the drawings are simplified for illustrative purposes and are not depicted to scale. Within the descriptions of the figures, similar elements are provided similar names and reference numerals as those of the previous figure(s). The specific numerals assigned to the elements are provided solely to aid in the description and are not meant to imply any limitations (structural or functional) on the invention.

The appended drawings illustrate exemplary configurations of the invention and, as such, should not be considered as limiting the scope of the invention that may admit to other equally effective configurations. It is contemplated that features of one configuration may be beneficially incorporated in other configurations without further recitation.

### DETAILED DESCRIPTION

The embodiments of the disclosure will be best understood by reference to the drawings, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the system and method of the disclosure, as represented in the Figures is not intended to limit the scope of the disclosure, as claimed, but is merely representative of possible embodiments of the disclosure.

In accordance with embodiments of the present invention, a method of risk stratification for contagious disease is provided. An object of the invention is to enable employers, local, and federal agencies to implement a testing plan to have citizens cleared to return to work during the COVID-19 pandemic. In one embodiment, the plan consists of three tiers to demonstrate employee risk or employee protection from COVID-19. Particularly, employees that are essential to the economy are able to get priority testing to ensure that they are safe to work. In one embodiment, qualitative testing will be by both reverse transcriptase polymerase chain reaction (RT-PCR) test and by antibody Immunoglobulin M (IgM) and Immunoglobulin G (IgG) assays. Employees who have had exposure to COVID-19 will get antibody routine monitoring by fingerstick and also by serial antibody titers. Quantitative testing using data of titers in patients exposed to and recovering from SARS-Covland MERS is used to measure benchmarks of ongoing protection against COVID-19. Serum IgG values of greater than 640 mg/dL (range 106 to 45816 mg/dL) would appear to be protective based on the range seen among the (OC43-106 to 8392, HKU1-127 to 45816, and SARS CoV1-160->640), neutralization titers of greater than 40 Iu/mL (range among the betacoronaviruses 20-160 Iu/mL), and binding titers of greater than 1:1000. Shen et. al recently demonstrated that the introduction of convalescent serum at a binding titer of 1:1000 and neutralization titers of >40 to critically ill patients infected with COVID-19 resulted in considerable improvement in symptoms. The method adjusts the benchmarks as public health data is compiled for COVID-19.

Patients that are not currently displaying any symptoms may represent more than 25% of those that are infected with COVID-19. Employees that are currently symptomatic for respiratory infection should remain at home until a definitive diagnosis is presented. It is therefore important that all essential employees and those that are interested in returning to work be tested by RT-PCR, qualitative antibody and quantitative antibody assays for circulating IgM and IgG. Embodiments of the present invention are operable to test for COVID-19 and reflex for other respiratory infections that can be addressed prior to an employee returning to work.

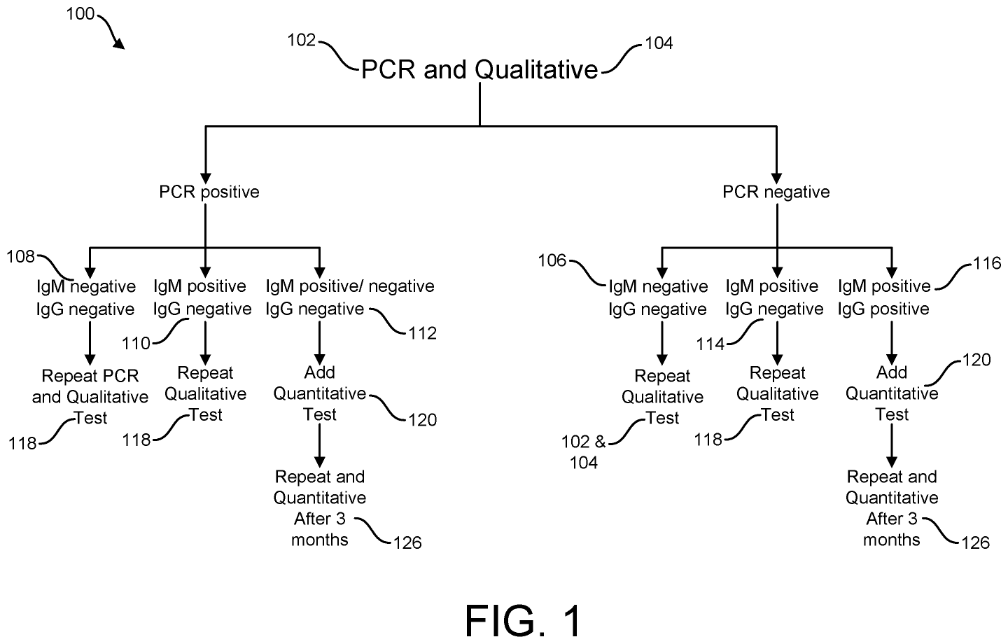

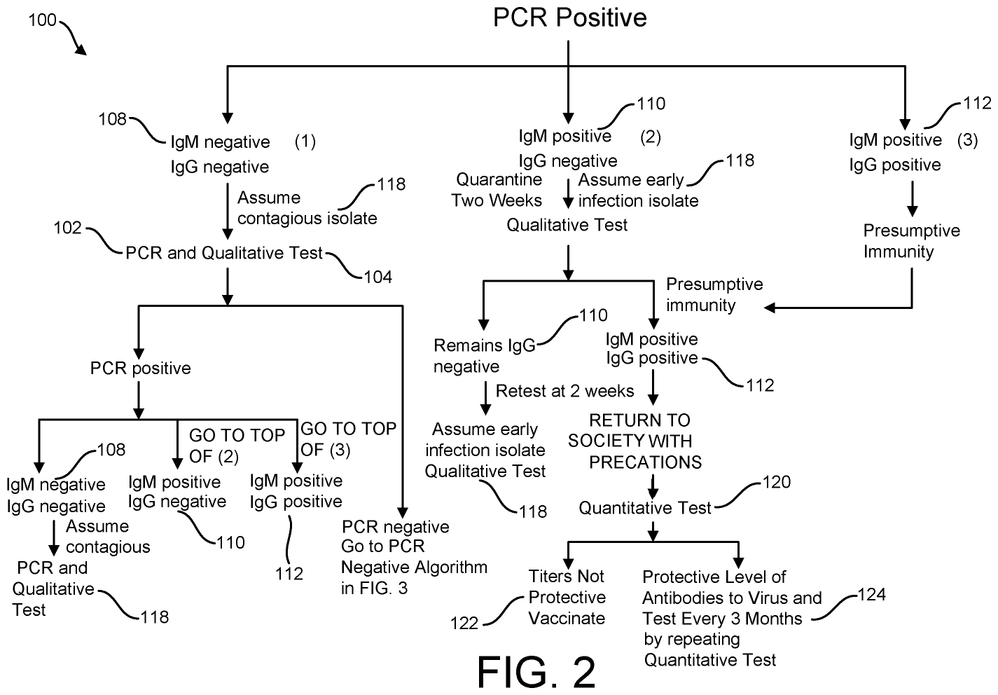

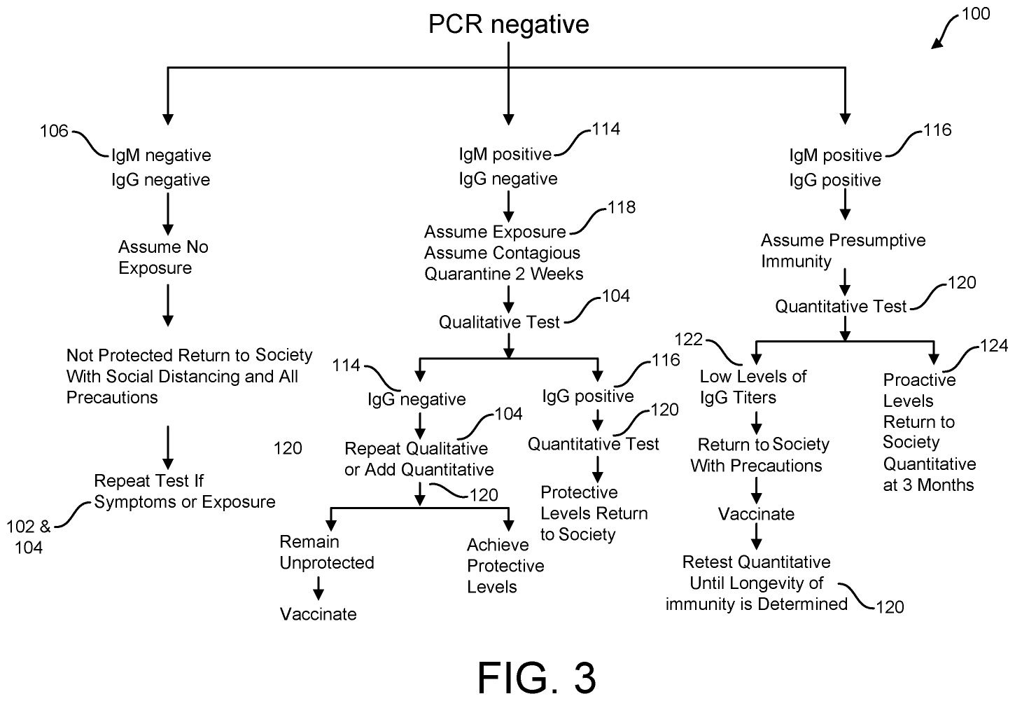

In accordance with embodiments of the invention, a method 100 of risk stratification for contagious disease is provided. As illustrated in FIGS. 1-3, the method includes a step (a) 102 of performing a reverse transcriptase polymerase chain reaction (RT-PCR) test on a patient to determine the presence of a viral infection. The viral infection may be the novel coronavirus that causes COVID-19 or SARS-Cov2, for example. FIG. 2 illustrates an algorithm of the method for patients that tested positive for COVID-19. FIG. 3 illustrates an algorithm of the method for patients that tested negative for COVID-19. The method includes a step (b) 104 of performing qualitative tests for the presence of antibody Immunoglobulin M (IgM) and Immunoglobulin G (IgG) assays. In step (c) 106, if the RT-PCR test in step (a) 102 is negative and the IgM and IgG tests in step (b) 104 are negative, the method 100 informs the patient that the patient has not been exposed to the virus, has not built any antibodies, does not have immunity, should not return to society, and repeat step (a) 102 and step (b) 104 every two weeks. Testing every two weeks may be repeated until the pandemic concludes, a vaccine is release, or herd immunity is established, or the patient has tested positive for protective immunity as a result of the following steps, for example. In step (d) 108, if the RT-PCR test in step (a) 102 is positive and the IgM and IgG tests in step (b) 104 are negative, the method 100 informs the patient that the patient is contagious, does not have immunity, has not produced antibodies, and moves to step (i) 118. In step (e) 110, if the RT-PCR test in step (a) 102 is positive, the IgM test in step (b) 104 is positive, and the IgG test in step (b) 104 is negative, the method 100 informs the patient that the patient is contagious, has begun producing antibodies, does not have immunity, and moves to step (i) 118. In step (f) 112, if the RT-PCR test in step (a) 102 is positive and the IgM and IgG tests in step (b) 104 are positive, the method 100 informs the patient that the patient is potentially contagious, has begun producing antibodies, and performs steps (j) 120 through (l) 124. In step (g) 114, if the RT-PCR test in step (a) 102 is negative, the IgM test in step (b) 104 is positive, and the IgG test in step (b) 104 is negative, the method 100 informs the patient that the patient has been exposed to the virus, is not contagious, has begun producing antibodies, does not have immunity, and performs step (i) 118. In step (h) 116, if the RT-PCR test in step (a) 102 is negative and the IgM and IgG tests in step (b) 104 are positive, the method 100 informs the patient that the patient has been exposed to the virus, is not contagious, and performs steps (j) 120 through (l) 124. The method 100 includes a step (i) 118 of recommending isolation for the patient for a first period of time, and repeating steps (a) 102 through (b) 104 upon the conclusion of the first period of time. The method 100 includes step (j) 120 of quantitative testing to determine the patient's level of titers of IgG. In step (k) 122, if the patient's level of IgG titers tested in step (j) 120 is low, such as below 640 mg/dL, for example, the method 100 recommends the patient refrain from reentry into society for a second period of time to allow for further production of IgG antibodies, and repeat steps (j) 120 through (l) 124 upon the conclusion of the second period of time. In step (l) 124, if the patient's level of IgG titers tested in step (j) 120 is high, such as above 640 mg/dL, for example, the method 100 informs the patient that the patient is immune, and recommends the patient return to society.

In one embodiment, the method 100 further includes the step 126 of testing the patient's IgM and IgG levels at three-month or six-month intervals, for example from the return to society to track immunity.

In one embodiment of the method 100, the patient is assigned in steps (c) 106 through (h) 116 a color and status level number indicate level of readiness to return to society. The color and status level number are displayed on a mobile device through a mobile application. For example, the color and status level number may be verified through a QR code displayed through the mobile application. In one embodiment, a centralized data process server hosts a database of patient test results and is operable to verify the status level and provide certification of authentication upon request from a mobile device, enabling patients to show a verified certificate to law enforcement or employers, for example. As the number of patients tested increases, the server may analyze test results and immunity levels, compare the levels with public health data, incorporate data from multiple laboratories, and revise benchmark levels for immunity.

In one embodiment of the method 100, the patient is assigned the color yellow and status level number 1 in step (c) 106, the color red and status level number 2 in step (d) 108, the color orange and status level number 3 in step (e) 110, the color orange and status level number 3 in step (f) 112 and remains level 3 until the PCR test is negative, the color blue and status level number 4 in step (g) 114, the color green and status level number 5 in step (d) 108, and the colors red, white, and blue and status level number 6 in step (l) 124.

In one embodiment, each scenario of testing will include serial monitoring of antibody titers every quarter to better define the longevity and quality of the antibody response. Should antibody titers drop in a patient below the accepted level, a boost via vaccination (spike and or nucleocapsid antigens) may be warranted to provide continued protection.

Exemplary tiers of testing:

Tier 1: RT-PCR in step (a) 102 is performed for the currently approved COVID-19 markers spike (S), nucleocapsid (N) and the orf-lab. RT-PCR is performed in a CLIA certified laboratory with skill in the testing and analysis of infectious diseases. RT-PCR testing is also available for symptomatic patients who are NEGATIVE for COVID-19 to determine specifically what respiratory pathogen is eliciting symptoms.

Tier 2: Qualitative antibody monitoring for IgM and IgG in step (b) 104 performed by venous blood draw or finger stick assay. IgM is pentameric antibody that is the first to be produced in the response to infection and the first to diminish. Over the first several weeks of an infection IgM is the predominant immunoglobulin present in the serum. Following IgM, IgG begins to take over as the predominant immunoglobulin circulating in the blood and can maintain levels for a long period of time. A point of care device, such as a lateral flow immunoassay, will be used to measure the presence or absence of IgM to indicate early infection and IgG to indicate late and convalescence.

Tier 3: Quantitative antibody titers will be monitored in step (j) 120 via blood draw to demonstrate that the employee has humoral protection from future infection. Appropriate protective IgG titers and levels are disclosed in the references with respect to the other coronavirus infections. The references disclose that the serum geometric mean titers for the betacoronaviruses is as follows. In the study by Gorse et. al, the mean geometric titer (GMT) for OC43 was 1235 (range of 127-45816), the GMT for HKU1 was 466 (range of 106-8392), and SARS Cov1 GMT was 320 (range of 160->640). Serial monitoring of employees every 3-6 months can inform on the continued protection of the workforce. As vaccination becomes available, patients with low levels of protective antibody can be boosted. Tier 3 testing would follow vaccination after a suitable period of time to determine that the vaccination was effective at increasing the serum IgG titers. Also, it can inform on who may need to remain as a remote employee should any employees display suboptimal IgG titers.

The benefits of this exemplary three-tier program extend beyond just reigniting the economy and returning employees to work. This method will also facilitate the accumulation of large data sets on the nature of seroconversion, the rates of PCR positive and negative results, the magnitude and duration of the serum antibody protection and potentially insight on antigen drift of the virus overtime. These data can then be used for drug development, vaccine refinement and a variety of other studies on the mechanisms and dynamics of COVID-19 infection and recovery.

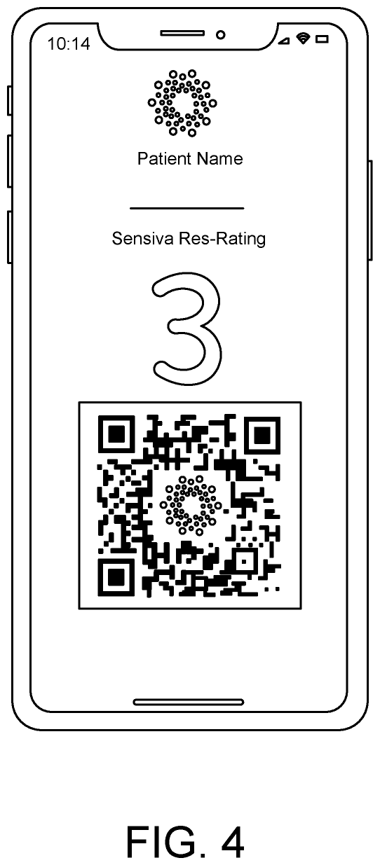

One embodiment of the invention includes a rating system generated based on a nasal phyargeal sample, blood antibody levels, blood quantitative tests and the method of risk stratification for contagious disease as disclosed herein determine how immune the patient is to COVID-19. The patient's ranking may be displayed on a smart phone with color coding, for example, and may be verified by QR code, for example, as illustrated in FIG. 4. Disclosed herein in an exemplary rating system using Levels 1 through 6 to indicate the patient's readiness to return to the public.

| Level | Test Results | Status |
|---|---|---|
| Level 1 | PCR−, IgM−, IgG− | NO EXPOSURE |
| Level 2 | PCR+, IgM−, IgG− | CONTAGIOUS |
| Level 3 | PCR+, IgM+, IgG− | CONTAGIOUS |
| Level 4 | PCR−, IgM+, IgG− | NOT CONTAGIOUS, |
| | | NOT PROTECTED YET |
| Level 5 | PCR−, IgM+, IgG+ | PRESUMPTIVE |
| | | PROTECTIVE IMMUNITY |
| Level 6 | IgM+, IgG+ | CONTINUED QUANTITATIVE |
| | | ANTIBODY TITER TESTING |

Level 1, which may be color coded as yellow, patient has no signs of ever being exposed to COVID-19 and has developed no protective immunity. Patient should continue social distancing and carefully follow all necessary precautions.

Level 2, which may be color coded as red, patient has tested positive for COVID-19 and has no protective immunity whatsoever. Patient should consider his or herself highly contagious and consult with their physician immediately. Patient should seek immediate medical attention if the illness progresses.

Level 3, which may be color coded as orange, patient has tested positive for COVID-19 and is starting to develop some immunity but should consider his or herself highly contagious until another test is completed in two weeks. Patient should seek immediate medical attention if the illness progresses.

Level 4, which may be color coded as blue, patient has been exposed to COVID-19 and developed some immunity. Patient has not yet developed full protective immunity and should re-test in two weeks. Patient should continue precautions such as social distancing.

Level 5, which may be color coded as green, patient has been exposed to COVID-19 and has developed some protective immunity. Patient can now return to work, get back to living life, and follow routine guidelines for protection.

Level 6, which may be color coded red, white and blue, patient has been exposed to COVID-19 and has developed protective immunity that can be measured quantitatively by antibody titer testing at three or six month intervals.

In one embodiment, a predictive model can be developed and revised at intervals, such as at three months intervals, relative to data provided by public health professionals and independent laboratories in making policy decisions based on evidence of immunity of titer levels quantitatively measured in patients. Using artificial intelligence, this model can be more accurate and more valuable as more data is added. The revisions of the algorithm with the data accumulation from labs, public health sources, and other sources will allow for informed public policy for COVID-19 and future pandemic response.

In one embodiment, a test kit includes the tools required for self-collection including a Nasopharyngeal swab kit for the PCR test and an LFD test kit for the antigen/antibody test. Detailed collection instructions with QR codes that link directly to collection videos are included.

In one embodiment, serum titers for healthcare and front-line personnel are greater than the 640 mg/dL level for all patients. Continued monitoring will be essential to the long-term protection of our healthcare community. The references regarding SARS and MERS show that over a period of 2-6 years the circulating antibody diminishes. Based on this data, the algorithm continues to test healthcare personnel on a regular basis as the information continues to be accumulated as to the longevity of the antibody response from COVID-19 infection.

1. A method of risk stratification for contagious disease, comprising:
(a) performing a reverse transcriptase polymerase chain reaction (RT-PCR) test on a patient to determine the presence of a viral infection;
(b) performing tests for the presence of antibody Immunoglobulin M (IgM) and Immunoglobulin G (IgG) assays; and
(c) assigning the patient a level of readiness to return to society corresponding to the combination of the results of the RT-PCR, IgM, and IgG tests in steps (a) and (b).

2. The method of claim 1, said method further comprising assigning the patient the color yellow and status level number 1 if the RT-PCR test in step (a) is negative and the IgM and IgG tests in step (b) are negative, informing the patient that the patient has not been exposed to the virus, has not built any antibodies, does not have immunity and should not return to society, and repeating step (a) and step (b) every two weeks.

3. The method of claim 1, said method further comprising assigning the patient the color red and status level number 2 if the RT-PCR test in step (a) is positive and the IgM and IgG tests in step (b) are negative, informing the patient that the patient is contagious, does not have immunity, has not produced antibodies, and recommending isolation for the patient for a first period of time, and repeating steps (a) through (b) upon the conclusion of the first period of time.

4. The method of claim 1, said method further comprising assigning the patient the color orange and status level number 3 if the RT-PCR test in step (a) is positive, the IgM test in step (b) is positive, and the IgG test in step (b) is negative, informing the patient that the patient is contagious, has begun producing antibodies, does not have immunity, and recommending isolation for the patient for a first period of time, and repeating steps (a) through (b) upon the conclusion of the first period of time.

5. The method of claim 1, said method further comprising assigning the patient the color blue and status level number 4 if the RT-PCR test in step (a) is negative, the IgM test in step (b) is positive, and the IgG test in step (b) is negative, informing the patient that the patient has been exposed to the virus, is not contagious, has begun producing antibodies, does not have immunity, and recommending isolation for the patient for a first period of time, and repeating steps (a) through (b) upon the conclusion of the first period of time.

6. The method of claim 1, said method further comprising assigning the patient the color green and status level number 5 if the RT-PCR test in step (a) is negative, the IgM test in step (b) is positive, and the IgG test in step (b) is positive, informing the patient that the patient has been exposed to the virus, is not contagious, has begun producing antibodies, has presumptive immunity, and recommending the patient return to society with precaution.

7. The method of claim 1, said method further comprising performing a quantitative test of IgG titers in patients that have tested positive for IgG antibodies with qualitative testing and assigning the patient the colors red, white and blue and status level number 6 if the patient's antibody titers are above a threshold for protective immunity, and informing the patient that the patient has protective immunity and can return to society without precautions, or if the patient's level of IgG titers tested are below the threshold for protective immunity, recommending the patient refrain from reentry into society for a second period of time to allow for further production of IgG antibodies, and retesting the level of titers upon the conclusion of the second period of time.

8. The method of claim 7, wherein the threshold for protective immunity of IgG titers is above 640 mg/dL.

9. The method of claim 1, further comprising assigning the patient a color and status level number indicating readiness to return to society corresponding to the combination of the results corresponding to the in step (c).

10. The method of claim 9, wherein the color level and status number are displayed on a mobile device through a mobile application.

11. The method of claim 10, wherein the color and status level number are verified through a QR code displayed through the mobile application.

12. A method of risk stratification for contagious disease, comprising:
(a) performing a reverse transcriptase polymerase chain reaction (RT-PCR) test on a patient to determine the presence of a viral infection;
(b) performing tests for the presence of antibody Immunoglobulin M (IgM) and Immunoglobulin G (IgG) assays;
(c) if the RT-PCR test in step (a) is negative and the IgM and IgG tests in step (b) are negative, inform the patient that the patient has not been exposed to the virus, has not built any antibodies, does not have immunity, should not return to society, and repeat step (a) and step (b) every two weeks;
(d) if the RT-PCR test in step (a) is positive and the IgM and IgG tests in step (b) are negative, informing the patient that the patient is contagious, does not have immunity, has not produced antibodies, and perform step (i);
(e) if the RT-PCR test in step (a) is positive, the IgM test in step (b) is positive, and the IgG test in step (b) is negative, informing the patient that the patient is contagious, has begun producing antibodies, does not have immunity, and perform step (i);
(f) if the RT-PCR test in step (a) is positive and the IgM and IgG tests in step (b) are positive, informing the patient that the patient is potentially still contagious, has begun producing antibodies, and perform steps (j) through (l);
(g) if the RT-PCR test in step (a) is negative, the IgM test in step (b) is positive, and the IgG test in step (b) is negative, informing the patient that the patient has been exposed to the virus, is not contagious, has begun producing antibodies, does not have immunity, and perform step (i);
(h) if the RT-PCR test in step (a) is negative and the IgM and IgG tests in step (b) are positive, informing the patient that the patient has been exposed to the virus, is not contagious, and perform steps (j) through (l);
(i) recommending isolation for the patient for a first period of time, and repeating steps (a) through (b) upon the conclusion of the first period of time;
(j) testing the patient's level of titers of IgG;
(k) if the patient's level of IgG titers tested in step (j) is less than 640 mg/dL, recommending the patient refrain from reentry into society for a second period of time to allow for further production of IgG antibodies, and repeat steps (j) through (l) upon the conclusion of the second period of time; and
(l) if the patient's level of IgG titers tested in step (j) is greater than 640 mg/dL, informing the patient that the patient is immune, and recommending the patient return to society.

13. The method of claim 12, said method further comprising the step of testing the patient's IgM and IgG levels at three-month intervals from the return to society to track immunity.

14. The method of claim 12, wherein the viral infection is the novel coronavirus that causes COVID-19.

15. The method of claim 12, said method further comprising assigning the patient in steps (c) through (h) a color and status level number indicate level of readiness to return to society.

16. The method of claim 15, wherein the color and status level number are displayed on a mobile device through a mobile application.

17. The method of claim 15, wherein the color and status level number are verified through a QR code displayed through the mobile application.

18. The method of claim 12, said method further comprising assigning the patient the color yellow and status level number 1 in step (c), the color red and status level number 2 in step (d), the color orange and status level number 3 in step (e), the color orange and status level number 3 in step (f) the color blue and status level number 4 in step (g), the color green and status level number 5 in step (k), and the colors red, white, and blue and status level number 6 in step (l).

19. The method of claim 12, wherein the level of titers in step (j) is compared to levels of titers in patients immune to SARS-Cov1.

20. A method of risk stratification for contagious disease, comprising:
(a) performing a reverse transcriptase polymerase chain reaction (RT-PCR) test on a patient to determine the presence of a viral infection;
(b) performing tests for the presence of antibody Immunoglobulin M (IgM) and Immunoglobulin G (IgG) assays;
(c) recommending isolation for the patient for a first period of time if the RT-PCR test in step (a) is positive and the IgM and IgG tests in step (b) are negative, and assigning a status level and color code to indicate the patient;
(d) performing tests for the presence of IgM and IgG assays after the end of the first period of time in step (c);
(e) repeating steps (c) and (d) until the test for IgG in step (d) is positive;
(f) testing the patient's level of titers of IgG when the IgG test in step (e) is positive;
(g) assigning the patient, a status level and a color code to indicate not protected if the level of titers tested in step (f) is low and recommending the patient refrain from reentry into society to allow for further production of IgG antibodies; and
(h) assigning the patient, a level status and color code to indicate protected if the level of titers tested in step (f) is high and recommending the patient return to society.

21. The method of claim 20, said method further comprising assigning the patient the color yellow and status level number 1 in if the RT-PCR test in step (a) is negative and the IgM and IgG tests in step (b) are negative, informing the patient that the patient has not been exposed to the virus, has not built any antibodies, does not have immunity, should not return to society, and repeating step (a) and step (b) every two weeks.

22. The method of claim 20, said method further comprising assigning the patient the color red and status level number 2 if the RT-PCR test in step (a) is positive and the IgM and IgG tests in step (b) are negative, informing the patient that the patient is contagious, does not have immunity, has not produced antibodies, and recommending isolation for the patient for a first period of time, and repeating steps (a) through (b) upon the conclusion of the first period of time.

23. The method of claim 20, said method further comprising assigning the patient the color orange and status level number 3 if the RT-PCR test in step (a) is positive, the IgM test in step (b) is positive, and the IgG test in step (b) is negative, informing the patient that the patient is contagious, has begun producing antibodies, does not have immunity, and recommending isolation for the patient for a first period of time, and repeating steps (a) through (b) upon the conclusion of the first period of time.

24. The method of claim 20, said method further comprising assigning the patient the color blue and status level number 4 if the RT-PCR test in step (a) is negative, the IgM test in step (b) is positive, and the IgG test in step (b) is negative, informing the patient that the patient has been exposed to the virus, is not contagious, has begun producing antibodies, does not have immunity, and recommending isolation for the patient for a first period of time, and repeating steps (a) through (b) upon the conclusion of the first period of time.

25. The method of claim 20, said method further comprising assigning the patient the color green and status level number 5 if the RT-PCR test in step (a) is negative, the IgM test in step (b) is positive, and the IgG test in step (b) is positive, informing the patient that the patient has been exposed to the virus, is not contagious, has begun producing antibodies, has presumptive immunity, and recommending the patient return to society with precaution.

26. The method of claim 20, said method further comprising a performing a quantitative test of IgG titers in patients that have tested positive for IgG antibodies with qualitative testing and assigning the colors red, white, and blue and status level number 6 if the patient's antibody titers are above a threshold for protective immunity, and informing the patient that the patient has protective immunity and can return to society without precautions, or if the patient's level of IgG titers tested in step is below the threshold for protective immunity, recommending the patient refrain from reentry into society for a second period of time to allow for further production of IgG antibodies, and retesting the level of titers upon the conclusion of the second period of time.

27. The method of claim 26, wherein the threshold for protective immunity of IgG titers is above 640 mg/dL.

28. The method of claim 20, said method further comprising the step of testing the patient's IgM and IgG levels at three-month intervals from the return to society to track immunity.

29. The method of claim 20, wherein the color and status level number are displayed on a mobile device through a mobile application.

30. The method of claim 29, wherein the color and status level number are verified through a QR code displayed through the mobile application.
